(19) United States
(12) Patent Application Publication
(10) Pub. No.: US 2025/0339576 A1
(43) Pub. Date: Nov. 6, 2025

(54) AIRCRAFT CABIN AIR IONIZER WITH BUILT IN ION MEASUREMENT SYSTEM

(71) Applicant: Hamilton Sundstrand Corporation, Charlotte, NC (US)

(72) Inventors: Srinivas Magaji Gundu, Bangalore (IN); Hemanth Raghav Gajendra, Bangalore (IN)

(21) Appl. No.: 19/076,250

(22) Filed: Mar. 11, 2025

(30) Foreign Application Priority Data
May 6, 2024 (IN) 202411035884

(51) Int. Cl. A61L 9/22; B01D 53/32; H01T 23/00

(52) U.S. Cl. CPC A61L 9/22; B01D 53/32; H01T 23/00; A61L 2209/11

(57) ABSTRACT

A system is provided including an ionizer configured to produce and emit charged air ions into an ionization region. The system includes an ion cell configured to output a first voltage corresponding to an ion density associated with the ionization region. The system includes a signal conditioning circuit configured to output a second voltage to the ionizer based on the first voltage. The ionizer is configured to maintain or adjust a setting associated with producing and emitting the charged air ions into the ionization region, based on the second voltage.

### CROSS-REFERENCE TO RELATED APPLICATIONS

This application claims the benefit of Indian Provisional Patent Application No. 202411035884 filed May 6, 2024, the disclosure of which is incorporated herein by reference in its entirety.

### BACKGROUND

Exemplary embodiments of the present disclosure pertain to the art of aircraft environmental control systems (ECS) and, in particular, to an aircraft cabin air ionizer with a built-in ion measurement system.

In general, the cabin air supply on most large commercial aircraft is provided using engine bleed air systems. There is evidence that some people experience acute symptoms due to a fume event or exposure to contaminants in aircraft cabin air. Further, it is desirable to ensure that the air is clean in order to remove or neutralize pathogens and other pollutants.

One approach to cleaning air is to provide an air ionization system in the aircraft and, in particular, in the ECS ducts and direct passenger air systems. In this manner the ionizer (possibly in combination with filters) can provide passengers with a fresher smelling cabin and cleaner air.

### BRIEF DESCRIPTION

Disclosed are air management systems and portions thereof, assemblies and methods. More particularly, the present disclosure provides for air management systems, assemblies and methods (e.g., for aircraft or the like), with the air management systems/assemblies having a cabin air ionizer for improved air quality.

Example embodiments of the present disclosure are directed to a system including: an ionizer configured to produce and emit charged air ions into an ionization region; an ion cell configured to output a first voltage corresponding to an ion density associated with the ionization region; and a signal conditioning circuit configured to output a second voltage to the ionizer based on the first voltage, wherein the ionizer is configured to maintain or adjust a setting associated with producing and emitting the charged air ions into the ionization region, based on the second voltage.

In any one or combination of the embodiments disclosed herein, the ion cell is configured to capture at least a portion of the charged air ions based on one or more third voltages provided by the signal conditioning circuit, wherein the signal conditioning circuit is configured to provide the one or more third voltages based on the first voltage.

In any one or combination of the embodiments disclosed herein: the signal conditioning circuit includes a programmable power source configured to provide one or more third voltages to the ion cell, based on a voltage setting.

In any one or combination of the embodiments disclosed herein: the ionizer is configured to provide the voltage setting; and the ionizer is configured to set or modify the voltage setting based on the second voltage.

In any one or combination of the embodiments disclosed herein: the signal conditioning circuit includes a programmable gain amplifier configured to generate the second voltage, based on the first voltage and a gain setting.

In any one or combination of the embodiments disclosed herein: the ionizer is configured to provide the gain setting; and the ionizer is configured to set or modify the gain setting based on the second voltage.

In any one or combination of the embodiments disclosed herein, the ionizer is configured to program or reprogram the signal conditioning circuit based on the second voltage.

In any one or combination of the embodiments disclosed herein, the system further includes a printed circuit board including a set of ground planes, wherein: the ion cell is disposed on the printed circuit board and coupled to the signal conditioning circuit via a signal trace included in the printed circuit board; and the signal trace is surrounded by the set of ground planes in a first direction parallel to a plane of the printed circuit board and a second direction perpendicular to the plane of the printed circuit board.

In any one or combination of the embodiments disclosed herein, a distance between the signal trace and at least one ground plane of the set of ground planes in the first direction is 10 mil or more.

In any one or combination of the embodiments disclosed herein, a thickness of the signal trace in the first direction is 10 mil or more.

In any one or combination of the embodiments disclosed herein: the ion cell includes is a cylindrical capacitor including an inner pin of a first voltage potential and an outer body of a second voltage potential; and the signal conditioning circuit is configured to set or adjust the first voltage potential and the second voltage potential based on the first voltage.

Example embodiments of the present disclosure are directed to an ionizer configured to: produce and emit charged air ions into an ionization region; and maintain or adjust a setting associated with producing and emitting the charged air ions into the ionization region, based on a voltage corresponding to an ion density associated with the ionization region.

In any one or combination of the embodiments disclosed herein, the ionizer is further configured to: set or modify a voltage setting associated with an ion cell in association with capturing at least a portion of the charged air ions.

Example embodiments of the present disclosure are directed to a method including: producing and emitting charged air ions into an ionization region; outputting a first voltage corresponding to an ion density associated with the ionization region; outputting a second voltage based on the first voltage; and maintaining or adjusting a setting associated with producing and emitting the charged air ions into the ionization region, based on the second voltage.

In any one or combination of the embodiments disclosed herein, the method further includes: setting or modifying a voltage setting associated with an ion cell in association with capturing at least a portion of the charged air ions, based on the second voltage; providing one or more third voltages to the ion cell, based on the voltage setting; and capturing at least a portion of the charged air ions based on the one or more third voltages.

In any one or combination of the embodiments disclosed herein, the method further includes: setting or modifying a setting associated with processing the first voltage, based on the second voltage.

Additional features and advantages are realized through the techniques of the present disclosure. Other embodiments and aspects of the disclosure are described in detail herein and are considered a part of the claimed technical concept. For a better understanding of the disclosure with the advantages and the features, refer to the description and to the drawings.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1A illustrates an example of a system in accordance with one or more embodiments of the present disclosure.

FIG. 1B illustrates an example of an ion cell in accordance with one or more embodiments of the present disclosure.

FIG. 2 illustrates an example block diagram of a system in accordance with one or more embodiments of the present disclosure.

FIG. 3 illustrates an example architecture of a system in accordance with one or more embodiments of the present disclosure.

FIG. 4 illustrates an example block diagram of a system and ion measurement/feedback provided by the system in accordance with one or more embodiments of the present disclosure.

FIG. 5 illustrates an example structure of a hybrid PCB in accordance with one or more embodiments of the present disclosure.

FIG. 6 illustrates an example architecture of a system in accordance with one or more embodiments of the present disclosure.

FIG. 7 illustrates an example flowchart of a method in accordance with one or more embodiments of the present disclosure.

### DETAILED DESCRIPTION

A detailed description of one or more embodiments of the disclosed apparatus and method are presented herein by way of exemplification and not limitation with reference to the Figures.

In general, current practice provides that some available air conditioning/management systems in aircraft cannot handle fume events (e.g., abnormal odors, smoke, haze or fumes in the cabin) that may arise from various internal or external sources, and some may be due to contamination of the bleed air supply (e.g., as a result of a failure of an oil seal in the engine). Moreover, some available air conditioning/management systems in aircraft cannot handle harmful viruses/bacteria efficiently.

The present disclosure provides improved air ECS systems having features capable of handling the poisonous/toxic chemicals/gases (e.g., carbon monoxide), climate relevant gases (e.g., methane), and/or harmful bacteria/viruses (e.g., SARS-COV-2), and improving the air quality inside the aircraft through the disclosed air ECS systems. Of course, embodiments do not need to include the entire ECS and some embodiments can be limited to the ionizer disclosed below.

As noted, some people experience acute symptoms due to a fume event or exposure to contaminants in conventional aircraft cabin air. Some of the chemical contaminants that are present during such events are irritants, and may cause itching or soreness of the eyes, nasal discharge, sore throat or coughing.

For example, turbine engine oil is an irritant and can include neurotoxic chemicals (e.g., tricresyl phosphate) Moreover, hydraulic fluid, although typically non-toxic in small quantities, can be extremely irritating to the eyes and skin, and can create a hazard to pilots during a fume event (but may cause no lasting damage). Furthermore, deicing fluid has a strong smell, but is not very irritating or toxic if inhaled (as opposed to the significant toxicity when ingested). In addition, other volatile organic compounds (VOC's) can be in aircraft cabins as well.

Disclosed herein is an ionizer. The ionizer can be used as part of aircraft ECS. The ionizer electrically mitigates VoC's, electrical fumes, smoke, molds, dust, malodor & pathogens, microbes both surface and airborne through ionization by ionizers

Currently, ionization performance is mostly defined in terms of ions generated—instantaneously. However, the inventors hereof have discovered that the ionization needs to be monitored effectively over longer timespans. However, in current scenarios, such monitoring is not possible as the ion generation units and ion measurement units are two distinct parts. Embodiments integrate the two parts into an ecosystem for efficient and effective performance monitoring. According to embodiments of the present disclosure, systems and techniques are described herein which support effective monitoring of ionization over longer timespans.

More limitations of the prior art may also be overcome by embodiments herein. In particular, current systems are non-feedback based. As such, this can limit the ionization product in terms of longer mean times between failures (MTBF), less cleaning/service status/product performance/Modularity/PoC installation ease/HMI access. According to embodiments of the present disclosure, systems and techniques are described herein which support closed loop monitoring of ionization.

Current ion emitters may be limited due to not having output control. This can cause the ion emitters to structurally degrade due to high voltage operation/due to environmental conditions (e.g., temperature, relative humidity, dust accumulation) without knowledge of the operator.

Further, current air ion counters (elements that quantify air ion density at a particular location and time instant) are separated from the current ionizers. These counters are expensive, bulky, and separated from the ionizer. Due to space constraints and construction, it is almost impossible to have a feedback mechanism in the current ionizer systems.

Embodiments herein can provide a compact feedback mechanism with built-in ion measurement system. The integrated mechanism may be able both measure ion count and provide the feedback to high voltage circuit for adjusting feedback gain/loss based on ion count. Further, the system may be a compact and inexpensive ion measurement system, address small signal measurement challenges handled by virtue of construction, quantify ion counts to be measured and controlled, and be configurable as per user requirements.

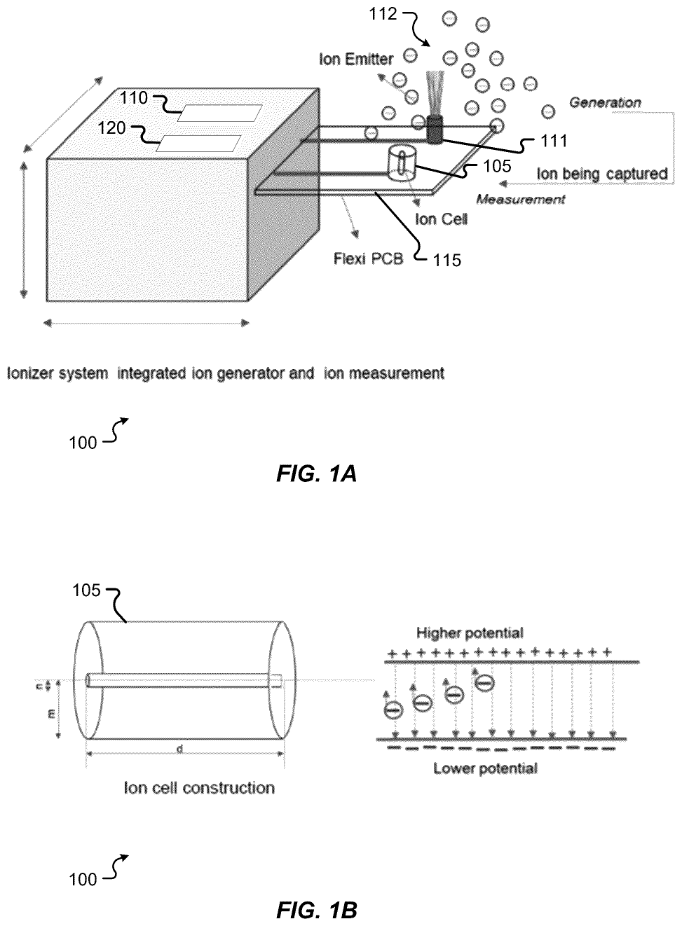

FIG. 1A illustrates an example of a system 100 in accordance with one or more embodiments of the present disclosure.

With reference to the system 100, ion measurement is performed by capture of generated ions, which provides adequate and consistent ion measurement. Ions are captured by allowing ions to flow through ion cell 105. The ion cell 105 is a concentric metal enclosure which resembles a cell and hence termed as an ion cell. The ion cell 105 is placed close (e.g., within about 5 cm to about 20 cm) to an ion emitter 111 of an ionizer 110 (also referred to herein as an ioniser) within an ionization region 112 (e.g., in which ion concentration is high with weak ionic wind). For example, the ionizer 110 (using the ion emitter 111) may produce and emit positively or negatively charged air ions, and the system 100 may support monitoring and capture of the charged air ions (and/or air molecules to which the charged air ions have become attached) in the ionization region 112. The ion emitter 111 may be coupled to (e.g., via a signal trace) or included in an ionizer 110.

The ionizer 110 may include circuitry supportive of features of the ionizer 110 described herein. The ionizer 110 may be a high-voltage direct current (HVDC) electric power transmission system configured to use direct current (DC) for electric power transmission.

The ionic wind driven ions pass through the ion cell 105 (e.g., through an electric field zone associated with the ion cell 105). In an embodiment, the ion cell 105 has a coaxially connected inner pin to input of input of an instrumentation amplifier, and an outer body of the ion cell 105 has a negative potential (for negative ion measurement). An example of the formed core construction of the ion cell 105 is illustrated at FIG. 1B, in which the ion cell 105 is a cylindrical capacitor (with electric field being perpendicular to the direction of ion flow). Ions are influenced by the electric field and move towards its opposite charged surface into the ion cell (see e.g., FIG. 1B). Typically, negative charged ions are drifted towards the positive charged plate. In some aspects, capacitance associated with the ion cell 105 is directly proportional to the length (m) of the cylinder and is inversely proportional to the log of the cell diameters (m, n), C α d/ln(m/n).

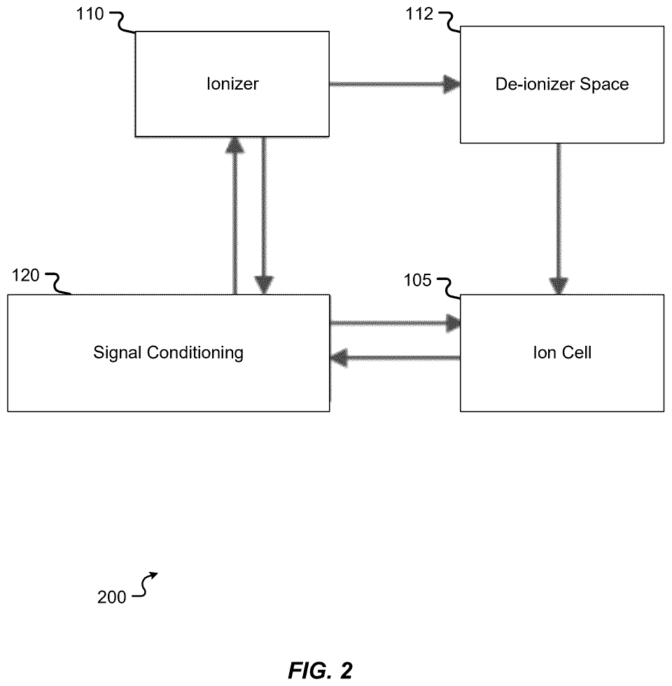

In embodiments herein, the ion cell 105 may provide a signal based on ions captured and passing through the ion cell 105, and the signal is passed through a hybrid PCB 115 and processed in a signal conditioning module later described herein. Embodiments of the present disclosure include programming the gain of the signal conditioning module based on a set ion density concentration, example aspects of which are later described herein. In an example, the systems and techniques described herein may include setting the PGA gain such that the PGA gain meets ADC dynamic range. See, e.g., FIGS. 2-7.

Embodiment of systems are shown in the figures. FIGS. 2 through 6 illustrate examples of the system 100 that support measuring ion concentration in accordance with one or more embodiments of the present disclosure.

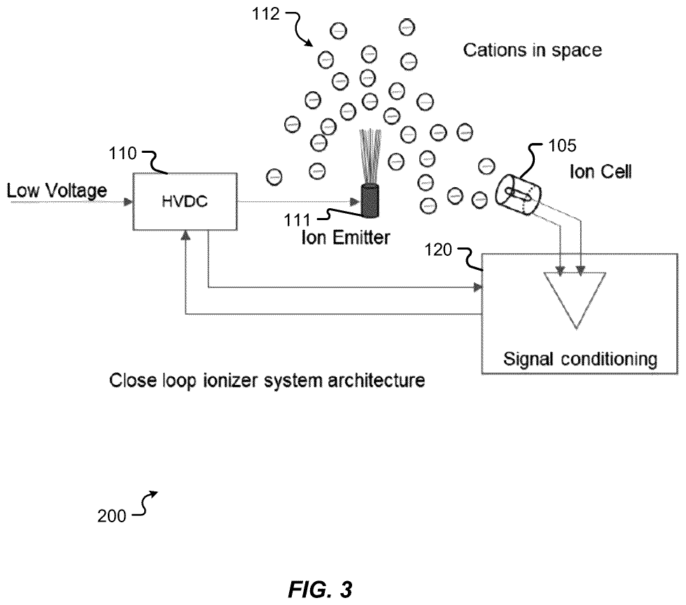

FIG. 2 illustrates an example block diagram of the system 100. The system 100 includes ionizer 110 with loop back to the signal conditioning circuit 120 and the ion cell 105 (e.g., via the signal conditioning circuit 120). FIG. 3 illustrates an example architecture of the system 100.

In an embodiment, the system 200 (an ion measurement system) forms a feedback path of a closed loop system (See FIGS. 2, 3, and 6). Measuring ion concentration in the ion field accurately is important for system operation. The system 200 provides increased accuracy associated with measuring ion concentration compared to other techniques.

The system 200 (ion measurement system) includes ion cell 105, a hybrid PCB 115 capable of propagating low power signals, and a signal conditioning circuit 120 capable of conditioning and converting the signal to a target power level. The signal conditioning circuit 120 may control or provide signals to high-voltage direct current (HVDC) electric power transmission circuitry 305 and/or the ion cell 105 in association with emitting ions into the ionization region 112. (see, e.g., FIGS. 2, 3 and 6). In some aspects, the system 200 may resemble or have characteristics of a Gerdien tube.

In an example, the ion cell 105, may be board-mounted to the hybrid PCB 115, and length and diameter of components (e.g., ion cell 105, hybrid PCB 115, and the like) of the system 100 may remain fixed. By varying applied voltage, the system 100 may achieve a target threshold (e.g., required threshold) of ions for consistent measurement using signal conditioning circuit 120. In an example, the signal conditioning circuit 120 may be a high gain signal conditioning circuit.

Ion concentration current is a very low signal which can be below the noise threshold. In accordance with one or more embodiments of the present disclosure, aspects of the system 100 may provide suitable signal transmission and conditioning for mitigating effects of noise (e.g., atmospheric noise). For example, the signal (i.e., ion concentration current) is prone to pick up atmospheric noise, and the hybrid PCB 115 may provide complete shielding of the signal,

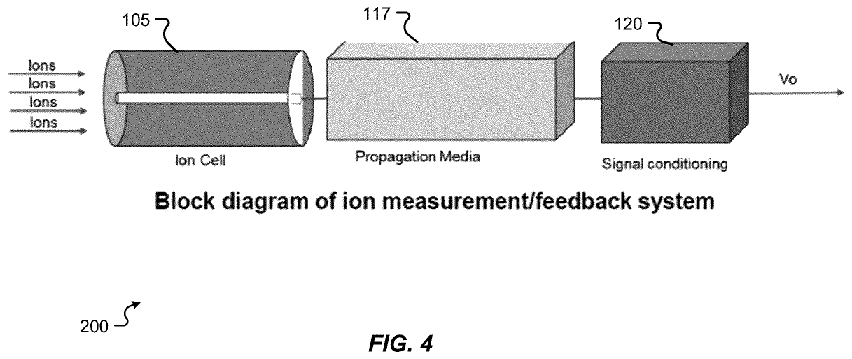

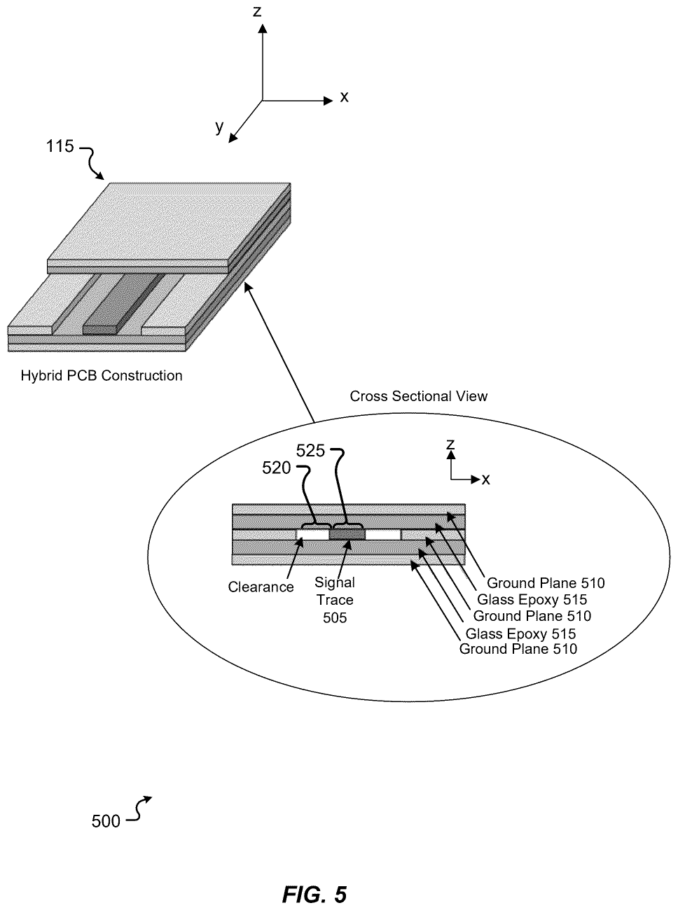

FIG. 4 illustrates an example block diagram of the system 100 and the ion measurement/feedback provided by the system 100. The ion cell 105 may provide signals to signal conditioning circuit 120 via propagation media 117. In some examples, the propagation media 117 may be signal traces formed on or in the hybrid PCB 115. Example aspects of the signal traces are later described herein with reference to FIG. 5.

FIG. 5 illustrates an example structure of the hybrid PCB 115 in accordance with one or more embodiments of the present disclosure. Hybrid PCB design guide lines (traces) are shown in FIG. 5. The hybrid PCB 115 may include signal trace 505, ground planes 510 (ground plane layers), and glass epoxy 515 (glass epoxy layers). The layer types, quantity, and configuration thereof are examples and are not limited to the example illustrated at FIG. 5.

In the example, signal trace 505 is routed as a stripline. The signal trace 505 supports shielding and transmission of signals (e.g., ion concentration current) described herein. The signal trace 505 is completely box shielded by having ground planes 510 at top and bottom layers of the hybrid PCB 115. In the inside layer, a transmitted signal has ground reference (ground plane 510) adjacent to the signal trace 505. The clearance 520 (e.g., in the x-direction) on either side of the signal trace 505 and the track thickness 525 (e.g., in the x-direction) of the signal trace 505 may support preventing or mitigating IR loss in the signal trace 505. In an example, the clearance 520 between the signal trace 505 and the ground plane 510 may be 10 mil or more. In an example, track thickness 525 of the signal trace 505 may be 10 mil or more.

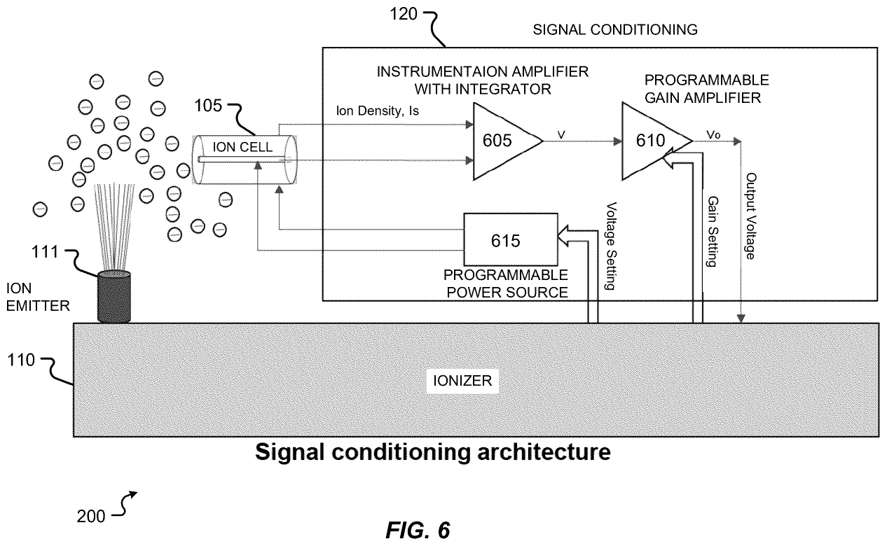

FIG. 6 illustrates an example architecture of the system 200 according to one or more embodiments. The signal conditioning circuit 120 (signal conditioner) may be programmable. For example, embodiments of the present disclosure support the system 100 programming the signal conditioning circuit 120 in association with meeting or satisfying the full-scale output range.

In the system of FIG. 6, the output of the ion cell 105 is output is fed to an instrumentation amplifier 605. In an example, the output of the ion cell 105 may include a voltage measurement across the outer body of the ion cell 105 and the inner pin of the ion cell 105. The voltage measurement may correspond to or represent an ion density (Is) (also referred to herein as an ion density concentration) of the ions in the ionization region 112 as determined by the ion cell 105.

The instrumentation amplifier 605 may include an integrator circuit. The instrumentation amplifier 605 may generate a voltage V. The voltage V output by the instrumentation amplifier 605 is fed to programmable gain amplifier (PGA) 610. In some embodiments, the instrumentation amplifier 605 in the first stage may have or support the following features: a high common-mode rejection (CMR) of greater than 80 dB, and an input noise kept to minimum (e.g., less than a threshold noise value). For example, the instrumentation amplifier 605 may have threshold noise value equal to less than 1 least significant bit (LSB) of the signal conditioning ADC.

The PGA 610 may be digitally programmable. In some embodiments, the PGA 610 may have a digitally programmable gain and output offset voltage. The PGA 610 may have a very wide gain range (e.g., from 10's to 1000's) and low offset voltage (e.g., in uV) (also referred to herein as an input offset voltage) over a wide temperature range. In an example, the temperature range may be −20 degrees Celsius to +70 degrees Celsius. The PGA 610 may generate and output a voltage Vo to the ionizer 110 based on the voltage V.

In one or more embodiments, the system 100 is programmable. For example, ion density has a relationship with the ionic wind and applied voltage based on type of ion cell 105. The ionizer 110 may be capable of programming the voltage to the ion cell 105. In some aspects, ionizer 110 may provide a voltage setting to the programmable power source 615 based on output voltage Vo, and the programmable power source 615 may provide a voltage(s) to the ion cell 105 based on the voltage setting.

The ion cell 105 may capture charged air ions (and/or air molecules to which the charged air ions have become attached) based on the voltage provided by the programmable power source 615. In an example, the values of the higher potential and lower potential associated with the ion cell 105 may be based on the voltage provided by the programmable power source 615.

The ionizer 110 may be capable of programming the PGA 610. For example, the ionizer 110 may provide a gain setting to the PGA 610 based on the voltage Vo. Accordingly, for example, based on the ion density determined from the ion cell 105, the ionizer 110 may program the gain of the PGA 610. Accordingly, for example, the system 100 may support programming the gain of the 610 in association with meeting a required full scale range of the ADC.

As described herein, aspects of the system 100 provide an ionizer 110 integrated with smart ion measurement. The system 100 may be implemented as a closed loop system, with programmable gain and a programmable power source to meet a wide dynamic range. The system 100 may be implemented with a smaller form factor, reduced cost, and reduce weight compared to other ion measurement devices. The system 100 may be implemented with a single controller. Accordingly, for example, the system 100 may be highly reliable compared to other ion measurement devices and systems.

The system 100 described herein provides functional benefits. For example, the ionizer 110 is controllable to meet various ion density based on end application. The system 100 is capable of providing stable ion count measurement.

Some comparative ion measurement devices may consume a space of 228 mm*170 mm*444 mm. In contrast, for example, implementation of the system 100 may consume a space of 40 mm*40 mm*6 mm, realizing space, weight, and costs savings.

As has been described herein, a system is provided including: an ionizer 110 configured to produce and emit charged air ions into an ionization region 112; an ion cell 105 configured to output a first voltage corresponding to an ion density associated with the ionization region 112; and a signal conditioning circuit 120 configured to output a second voltage to the ionizer 110 based on the first voltage. The ionizer 110 is configured to maintain or adjust a setting associated with producing and emitting the charged air ions into the ionization region 112, based on the second voltage.

The ion cell 105 is configured to capture at least a portion of the charged air ions based on one or more third voltages provided by the signal conditioning circuit 120, where the signal conditioning circuit 120 is configured to provide the one or more third voltages based on the first voltage.

The signal conditioning circuit 120 includes a programmable power source 615 configured to provide one or more third voltages to the ion cell 105, based on a voltage setting. The ionizer 110 is configured to provide the voltage setting. The ionizer 110 is configured to set or modify the voltage setting based on the second voltage.

The signal conditioning circuit 120 includes a programmable gain amplifier 610 configured to generate the second voltage, based on the first voltage and a gain setting. The ionizer 110 is configured to provide the gain setting. The ionizer 110 is configured to set or modify the gain setting based on the second voltage.

The ionizer 110 is configured to program or reprogram the signal conditioning circuit 120 based on the second voltage.

The system includes a printed circuit board 115 including a set of ground planes 510, where: the ion cell 105 is disposed on the printed circuit board 115 and coupled to the signal conditioning circuit 120 via a signal trace 505 included in the printed circuit board 115; and the signal trace 505 is surrounded by the set of ground planes 510 in a first direction parallel to a plane of the printed circuit board 115 and a second direction perpendicular to the plane of the printed circuit board 115.

A distance between the signal trace 505 and at least one ground plane 510 of the set of ground planes 510 in the first direction is 10 mil or more.

A thickness of the signal trace 505 in the first direction is 10 mil or more.

The ion cell 105 includes is a cylindrical capacitor including an inner pin of a first voltage potential and an outer body of a second voltage potential; and the signal conditioning circuit 120 is configured to set or adjust the first voltage potential and the second voltage potential based on the first voltage.

As has been described herein, an ionizer 110 is provided which is configured to: produce and emit charged air ions into an ionization region 112; and maintain or adjust a setting associated with producing and emitting the charged air ions into the ionization region 112, based on a voltage corresponding to an ion density associated with the ionization region 112. The ionizer 110 is configured to set or modify a voltage setting associated with an ion cell 105 in association with capturing at least a portion of the charged air ions.

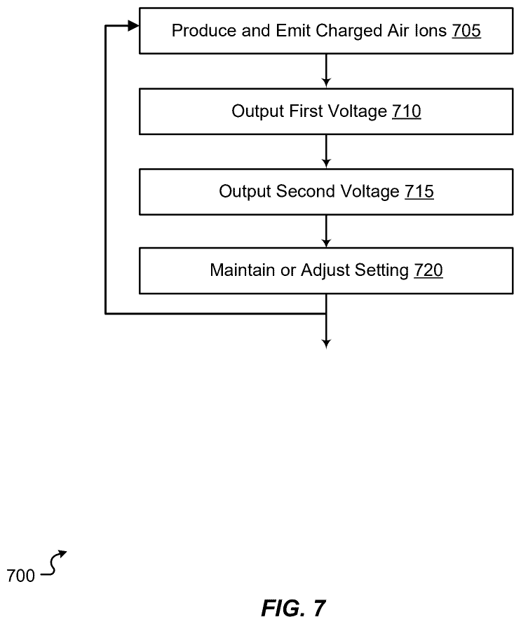

FIG. 7 illustrates an example flowchart of a method 700 in accordance with one or more embodiments of the present disclosure. The method 700 may be implemented by the example aspects of a system 100 described herein.

At 705, the method 700 includes producing and emitting charged air ions into an ionization region.

At 710, the method 700 includes outputting a first voltage corresponding to an ion density associated with the ionization region.

At 715, the method 700 includes outputting a second voltage based on the first voltage.

At 720, the method 700 includes maintaining or adjusting a setting associated with producing and emitting the charged air ions into the ionization region, based on the second voltage.

In some aspects, the method 700 may include: setting or modifying a voltage setting associated with an ion cell in association with capturing at least a portion of the charged air ions, based on the second voltage; providing one or more third voltages to the ion cell, based on the voltage setting; and capturing at least a portion of the charged air ions based on the one or more third voltages.

In some aspects, the method 700 may include setting or modifying a setting associated with processing the first voltage, based on the second voltage.

In the descriptions of the flowcharts herein, the operations may be performed in a different order than the order shown, or the operations may be performed in different orders or at different times. Certain operations may also be left out of the flowcharts, one or more operations may be repeated, or other operations may be added to the flowcharts.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A system comprising:
an ionizer configured to produce and emit charged air ions into an ionization region;
an ion cell configured to output a first voltage corresponding to an ion density associated with the ionization region; and
a signal conditioning circuit configured to output a second voltage to the ionizer based on the first voltage,
wherein the ionizer is configured to maintain or adjust a setting associated with producing and emitting the charged air ions into the ionization region, based on the second voltage.

2. The system of claim 1, wherein the ion cell is configured to capture at least a portion of the charged air ions based on one or more third voltages provided by the signal conditioning circuit,
wherein the signal conditioning circuit is configured to provide the one or more third voltages based on the first voltage.

3. The system of claim 1, wherein:
the signal conditioning circuit comprises a programmable power source configured to provide one or more third voltages to the ion cell, based on a voltage setting.

4. The system of claim 3, wherein:
the ionizer is configured to provide the voltage setting; and
the ionizer is configured to set or modify the voltage setting based on the second voltage.

5. The system of claim 1, wherein:
the signal conditioning circuit comprises a programmable gain amplifier configured to generate the second voltage, based on the first voltage and a gain setting.

6. The system of claim 5, wherein:
the ionizer is configured to provide the gain setting; and
the ionizer is configured to set or modify the gain setting based on the second voltage.

7. The system of claim 1, wherein the ionizer is configured to program or reprogram the signal conditioning circuit based on the second voltage.

8. The system of claim 1, further comprising a printed circuit board comprising a set of ground planes, wherein:
the ion cell is disposed on the printed circuit board and coupled to the signal conditioning circuit via a signal trace comprised in the printed circuit board; and
the signal trace is surrounded by the set of ground planes in a first direction parallel to a plane of the printed circuit board and a second direction perpendicular to the plane of the printed circuit board.

9. The system of claim 8, wherein a distance between the signal trace and at least one ground plane of the set of ground planes in the first direction is 10 mil or more.

10. The system of claim 8, wherein a thickness of the signal trace in the first direction is 10 mil or more.

11. The system of claim 1, wherein:
the ion cell comprises is a cylindrical capacitor comprising an inner pin of a first voltage potential and an outer body of a second voltage potential; and
the signal conditioning circuit is configured to set or adjust the first voltage potential and the second voltage potential based on the first voltage.

2. An ionizer configured to:
produce and emit charged air ions into an ionization region; and
maintain or adjust a setting associated with producing and emitting the charged air ions into the ionization region, based on a voltage corresponding to an ion density associated with the ionization region.

13. The ionizer of claim 12, further configured to:
set or modify a voltage setting associated with an ion cell in association with capturing at least a portion of the charged air ions.

3. A method comprising:
producing and emitting charged air ions into an ionization region;
outputting a first voltage corresponding to an ion density associated with the ionization region;
outputting a second voltage based on the first voltage; and
maintaining or adjusting a setting associated with producing and emitting the charged air ions into the ionization region, based on the second voltage.

15. The method of claim 14, further comprising:
setting or modifying a voltage setting associated with an ion cell in association with capturing at least a portion of the charged air ions, based on the second voltage;
providing one or more third voltages to the ion cell, based on the voltage setting; and
capturing at least a portion of the charged air ions based on the one or more third voltages.

16. The method of claim 14, further comprising:
setting or modifying a setting associated with processing the first voltage, based on the second voltage.
